(19) United States
(12) Patent Application Publication
(10) Pub. No.: US 2013/0337548 A1
(43) Pub. Date: Dec. 19, 2013

(54) Rotating Bioreactor

(71) Applicants: Ronald Sims, Logan, UT (US); Reese Thompson, Nibley, UT (US); Steven T. Goldsmith, Salt Lake City, UT (US); Brad Hansen, Salt Lake City, UT (US); Terence Smith, Smithfield, UT (US); Logan Christenson, Charlottesville, VA (US)

(72) Inventors: Ronald Sims, Logan, UT (US); Reese Thompson, Nibley, UT (US); Steven T. Goldsmith, Salt Lake City, UT (US); Brad Hansen, Salt Lake City, UT (US); Terence Smith, Smithfield, UT (US); Logan Christenson, Charlottesville, VA (US)

(73) Assignee: UTAH STATE UNIVERSITY, North Logan, UT (US)

(21) Appl. No.: 13/952,469

(22) Filed: Jul. 26, 2013

Related U.S. Application Data
(63) Continuation-in-part of application No. 13/040,364, filed on Mar. 4, 2011.
(60) Provisional application No. 61/676,186, filed on Jul. 26, 2012.
(60) Provisional application No. 61/310,360, filed on Mar. 4, 2010.

(51) Int. Cl. C12N 1/12

(52) U.S. Cl. CPC C12N 1/12
USPC 435/257.1; 435/289.1

(57) ABSTRACT

A bioreactor, including at least one rotating drum, a substrate on the drum whereon a biofilm may grow, a rotational device rotating the drum and substrate, and a harvesting device.

### CROSS REFERENCE TO RELATED APPLICATIONS

This application is a continuation-in-part application of U.S. patent application Ser. No. 13/040,364, filed Mar. 4, 2011, which claims benefit under 35 U.S.C. §119(e) to U.S. Provisional Patent Application No. 61/310,360, filed Mar. 4, 2010. This application also claims benefit under 35 U.S.C. §119(e) to U.S. Provisional Patent Application No. 61/676,186, filed Jul. 26, 2012. Each of the above-referenced applications is incorporated herein by reference in its entirety.

### GOVERNMENT SPONSORED RESEARCH

This invention was made at least in part with government support under contract DE-EE003114 awarded by the Department of Energy. The government has certain rights in the invention.

### TECHNICAL FIELD

The present disclosure relates to materials, apparatuses, and methods for growing and harvesting biomass. Specifically, this disclosure relates to materials, apparatuses, and methods for harvesting algae.

### BACKGROUND

Excess nitrogen, phosphorus, and other nutrients or compounds in discharged wastewaters can lead to do :stream eutrophication and ecosystem damage. Advanced wastewater treatment technologies capable of removing these nutrients are expensive and often require the addition of chemical precipitants. Nitrogen (N) and phosphorus (P) can be removed naturally through biomass assimilation, but heterotrophic bacteria typically become carbon limited before removing all soluble N and P. Because microalgae are autotrophic, they can overcome this limitation and assimilate the remaining nutrients. In addition to the environmental benefits of harvesting algae grown during wastewater treatment, harvested microalgae are valuable as fertilizer, high-protein animal feed, and feedstock for the production of biofuels, including biodiesel and biomethane. Nutraceuticals, polymers, and other valuable products can be obtained from microalgae as well. Previously however, the realization of such benefits has been handicapped by an inability to find a reliable and cost effective apparatus and method of growing and harvesting the algae.

Methods of growing algae at large scale include open outdoor pond systems and closed tubular photobioreactors. The most common outdoor pond design is the high rate algal pond, or raceway pond. These are shallow ponds that circle a volume of nutrient rich water by means of a paddle wheel. Although relatively inexpensive to build, large plots of land are required and the resulting algae yields are lower than with closed reactors. Tubular photobioreactors can often achieve higher cell concentrations than open ponds, but suffer from high material cost and frequent cell death due to inefficient gas exchange. Biofouling of the reactor walls also decreases light penetration and cleaning becomes an issue as well. With both methods, the resulting solution of suspended microalgae is very dilute, necessitating high cost methods of separation.

In such methods, suspended microalgae must be removed from very dilute solutions and concentrated before further processing is possible. Current separation methods include filtration, sedimentation, centrifugation, dissolved air flotation, addition of electrolytes and polymers to induce coagulation and flocculation, and multiple combinations of these operations. Separation through filtration is difficult due to the small size of planktonic microalgae, and the sedimentation rate of algae is too slow for separation on a reasonable time scale. Dissolved air flotation requires high energy and high electrolyte and/or polymer addition to sufficiently flocculate microalgae. Centrifugation is currently the most common method used to separate algae from aqueous solutions; however, high upfront capital costs, power demand, and frequent maintenance make it uneconomical for large-scale use.

Existing biofilm reactors designed for the purpose of growing attached cultures include continuous stirred tank reactors with fibrous bed support, biofilm packed bed reactors, biofilm trickling bed reactors, and biofilm fluidized bed reactors. Such reactors use a porous support or small granules as substrata for cell attachment and biofilm growth. These reactor configurations are often used to treat wastewater or produce a secreted product, but are limited in that harvesting of the biomass or intracellular product is not possible without high cost.

### SUMMARY

The present disclosure in aspects and embodiments addresses these various needs and problems by providing materials, apparatuses, and methods for growing and harvesting biomass, specifically, algae. This disclosure describes a bioreactor, with associated materials and methods, which comprises at least one rotating drum, a substrate on the drum whereon a biofilm may grow, a rotational device rotating the drum and substrate, and a harvesting device.

### BRIEF DESCRIPTION OF THE DRAWINGS

FIG. 1 illustrates a bioreactor.

FIGS. 2a and 2b illustrate a drum (FIG. 2a is a plan view, FIG. 2b is a side elevation view).

FIGS. 3a and 3b illustrate bioreactor systems.

FIG. 4a-4c illustrate a guide component (FIG. 4a is an isometric view, 4b is a plan view, FIG. 4c is a side elevation view).

FIG. 5 is a flow diagram of an exemplary system.

FIGS. 6a-6d illustrate a harvesting device (FIG. 6a is a plan view, FIG. 6b is a front elevation view, FIG. 6c is a side elevation view, FIG. 6d is an isometric view).

FIGS. 7a-7d illustrate a collection device (FIG. 7a is a plan view, FIG. 7b is a side elevation view, FIG. 7c is a front elevation view, FIG. 7d is an isometric view).

FIG. 8 illustrates a tank.

FIG. 9 illustrates a bioreactor including harvesting and collection devices.

FIG. 10 illustrates a multiple drum bioreactor system.

FIG. 11 illustrates a bioreactor with a flotation frame.

FIG. 12 illustrates a bioreactor in a raceway.

FIG. 13 illustrates production data from an exemplary bioreactor.

### DETAILED DESCRIPTION

The present disclosure covers materials, apparatuses, kits, compositions, and associated methods for growing and harvesting biomass.

In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

### I. Growth Medium and Seed Culture

In embodiments, the biomass, which may be algae, is grown in a liquid medium. Any suitable liquid medium may be used. Different formulations of liquid medium will be used to produce different types of biomass.

The liquid medium may be a complex, defined, or selective growth medium. More specifically, the liquid medium may be a complex medium including, but not limited to complex dextrose based media, sea water media, domestic wastewater, municipal wastewater, industrial wastewater, surface runoff wastewater, soil extract media, or any natural water containing detectable amounts of phosphorus or nitrogen; or a defined medium, including, but not limited to Bristol's medium, Bolds Basal medium, Walne medium, Guillard's f medium, Blue-Green medium, D medium, DYIY medium, Jaworski's medium, K medium, MBL medium, Jorgensen's medium, and MLA medium; or a selective medium including, but not limited to minimal media based on specific nutrient auxotrophy, and selective media that incorporates antibiotics.

In embodiments, the medium may be specifically seeded with a seed culture including at least one microbe, or the liquid medium may contain microbes, such as microalgae. For example, if municipal wastewater is used in whole or part as the medium, then the medium will naturally contain numerous microbes and/or microalgae species.

Depending on the chosen liquid medium and seed culture, the resulting biofilm may be a mixed or pure culture and may comprise microalgae, cyanobacteria, nitrifying bacteria, heterotrophic bacteria, microscopic fungi, or any combination thereof.

In embodiments, seed cultures that are capable of growing as biofilms are preferred. For example, in addition to planktonic growth, microalgae are capable of growing as biofilms attached to surfaces. Algal biofilms, or periphyton, are able to remove nutrients from wastewater just as suspended algae, and harvested biofilms may then be processed into valuable products just as harvested suspended algae. When algae are grown as biofilms, the biomass is naturally concentrated and more easily harvested, leading to more direct removal and reduced downstream processing. The extracellular polymeric substance secreted by biofilms also increases the flocculation of associated suspended cells. Previously, however, there were no methods of growing and harvesting algal biofilms with any full-scale potential.

In addition to microalgae, other microorganisms are capable of growing as biofilms attached to surfaces. Biofilms are often complex mixed cultures containing microalgae, cyanobacteria, heterotrophic bacteria, nitrifying bacteria, microscopic fungi, and various combinations of these types of organisms. When grown as biofilms, the organisms' morphology and metabolism are often different than when the organism is suspended. These changes are often beneficial, and can include increased production of a desired product.

### II. Bioreactor

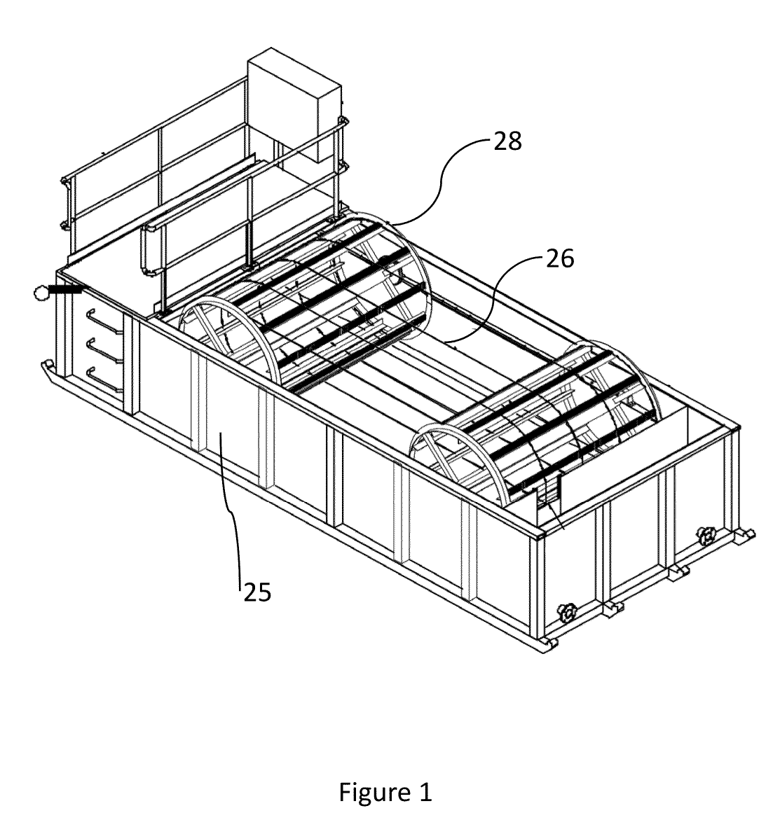

In embodiments, a bioreactor may be used to harvest biomass produced in the growth medium. The bioreactor may comprise at least one drum, a biofilm substrate, a rotational device driving the drum, and a harvesting apparatus. FIG. 1 illustrates an exemplary bioreactor.

### A. Drum

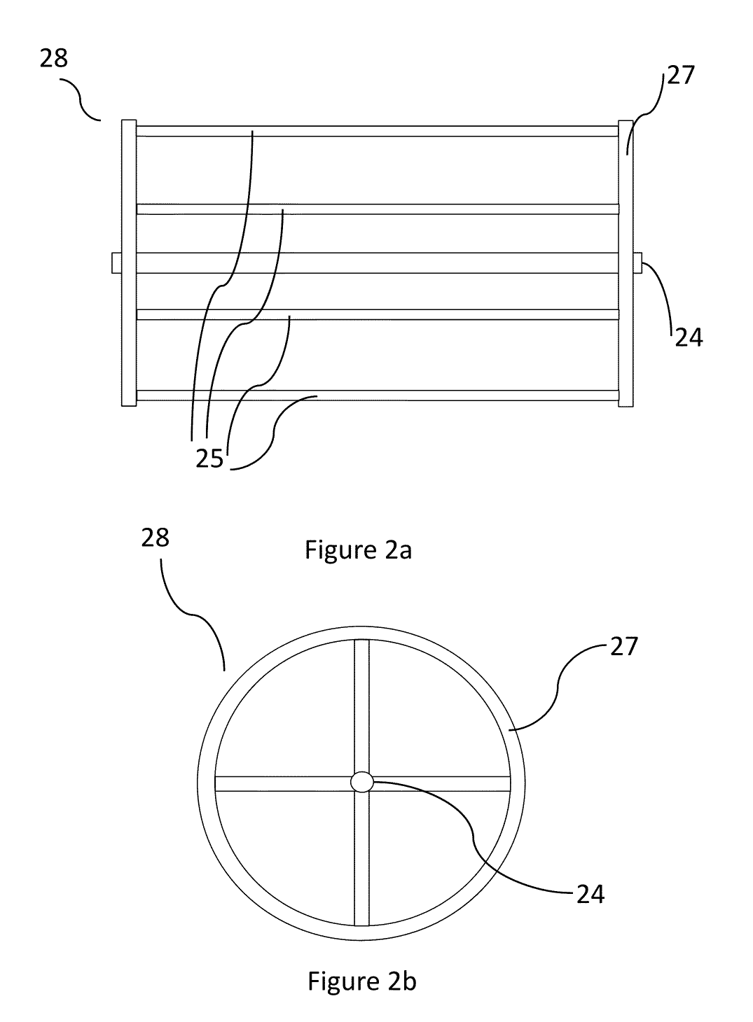

In embodiments, the bioreactor comprises at least one drum 28. The drum 28 is configured with at least two hubs 27 mechanically coupled about an axis 24. The drum 28 further comprises at least two struts 25 connecting the hubs and configured to support the substrate. An exemplary drum is illustrated in FIGS. 2a and 2b.

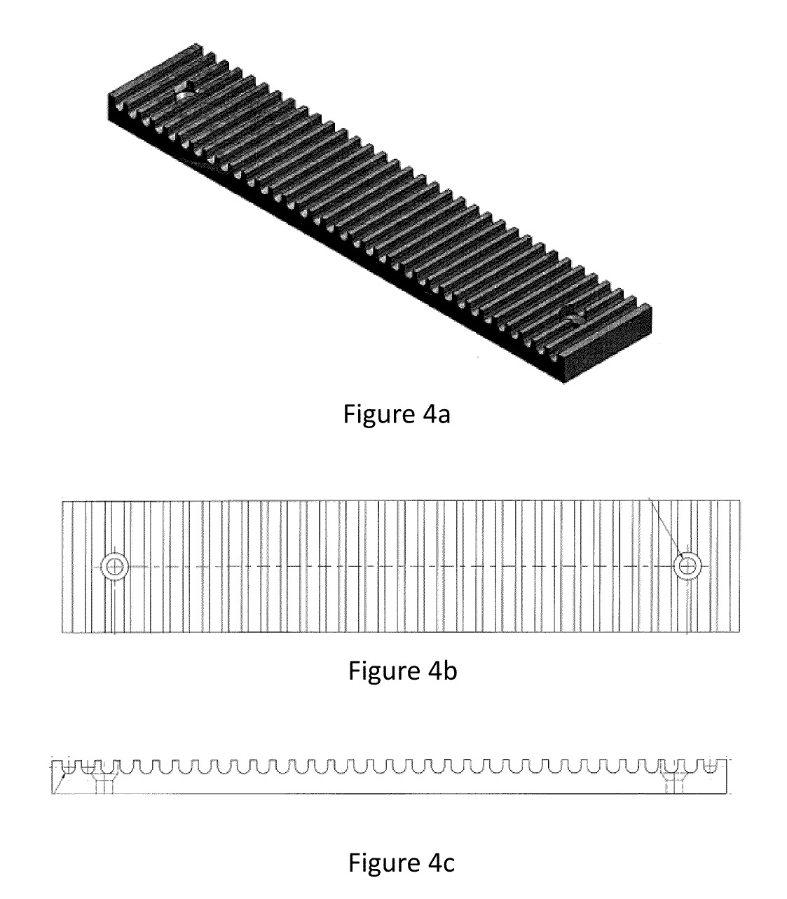

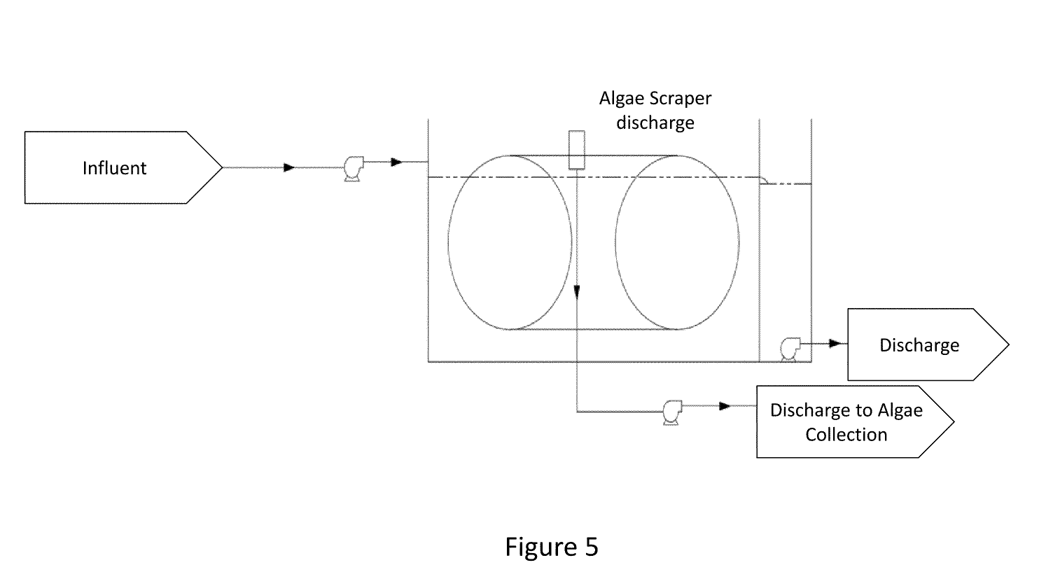

The struts 25 may be optionally configured with a guide component to facilitate the alignment of the substrate along the struts. The guide component may include scallops, ridges, groves, or other means of guiding/aligning the loops or coils of substrate. The guide component may be varied depending on the diameter or shape of the substrate. An exemplary guide component is illustrated in FIGS. 4a, 4b, and 4c. The guide component may be made of any suitable material, including, for example, metal, alloys, steel, Teflon, Teflon-coated steel, rubber, plastic, composites, or combinations thereof

The hubs 27 may be configured in any suitable shape to enable a substrate to be rotated into the growth medium and then out of the growth medium. This process provides for biofilm formation upon the substrate by providing nutrients and seed culture within the growth medium along with energy (light) and air (oxygen and other gases) when rotated out of the liquid medium. Exemplary hub shapes include a circular, oval, elliptical, polygonal, triangular, star, square, etc.

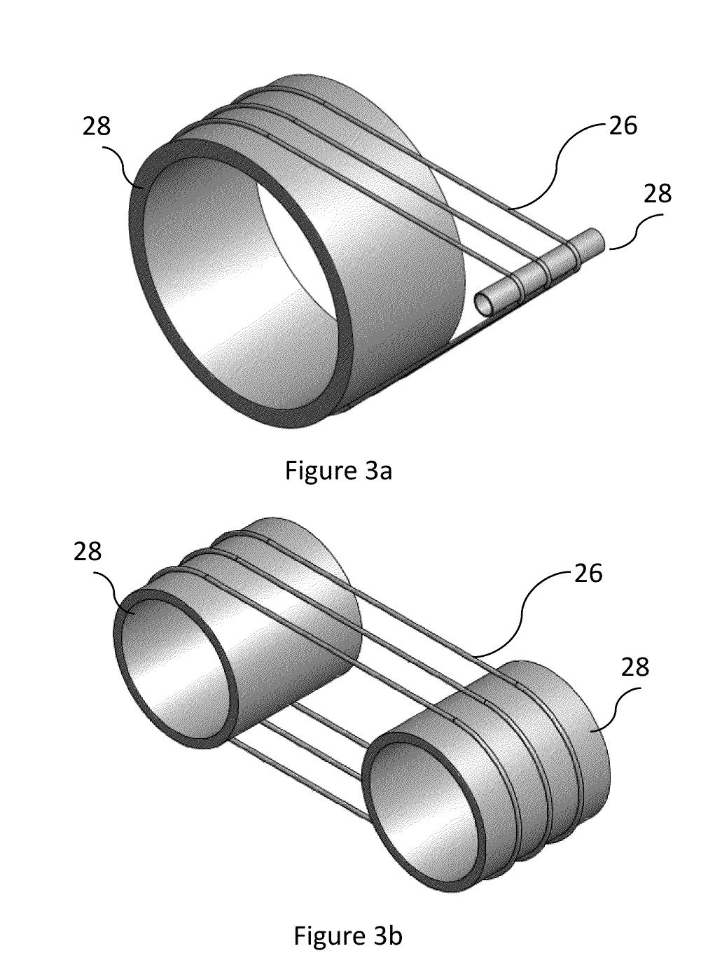

In embodiments employing multiple drums, the drums may be the same or different in size or design, as illustrated in FIGS. 3a and 3b. In multi-drum apparatuses, the drums may be placed in series to maximize biofilm growth conditions. Multi-drum systems may include at least two drums, but may include up to 5, 10, 50, 100, or 1000 drums in a single system.

### B. Biofilm Substrate

In embodiments, any suitable biofilm substrate 26 may be used. The substrate 26 may be in the form of a rope, cable, or belt. The substrate 26 may be configured as a single coil, as is illustrated in FIG. 9, or may be configured into multiple, separate loops, arranged in line around the drum(s), such as is illustrated in FIGS. 3a and 3b.

In embodiments employing a single coil of substrate, the substrate 26 may be spooled/coiled around the struts of the drum 28 or drums. One end of the substrate 26 may be attached to the drum 28 and wound around until the drum(s) may be sufficiently covered with the substrate. The free end of the substrate 26 may then be secured to a drum to keep the substrate from unwinding during rotation of the drum 28.

In embodiments employing separate loops arranged in a line, any number of loops necessary to cover the length of the struts may be used. The loops are placed around the struts next to each other to maximize the substrate surface area. The number of loops employed may vary depending on the diameter or width of the substrate and the size of the drum. The loops may be formed by joining two ends of the substrate.

The substrate material may be selected from cotton, jute, hemp, manila, silk, linen, sisal, silica, acrylic, polyester, nylon, polypropylene, polyethylene, polytetrafluroethylene, polymethylmethacrylate, polystyrene, polyvinyl chloride, any other non-rigid material capable of supporting biofilm growth, or any combination of one or more of the above materials. The substrate material is capable of performing in a wet environment (growth medium) and preferably is resistant to stretching in such an environment. In some embodiments, the substrate material may be selected from a rope with a cable core, which aids in stretch resistance and loop formation. When using a cable-core rope, the loop may be formed by tying, welding, mechanically attaching, or otherwise joining the cable-core ends. The cable core may be made of any suitable metal, such as aluminum, galvanized steel, copper, alloys, or stainless steel, though stainless steel is preferable for biofilm production.

The diameter of the substrate material may vary in size depending on the biofilm to be produced and the size of the bioreactor. Exemplary substrate material diameters include from about 2 mm to about 40 mm, such as about 5 mm to about 30 mm, about 6 mm to about 20, or about 8 mm to about 16 mm.

In embodiments employing a single coil of substrate, the substrate may be spooled/coiled around the struts of the drum or drums. One end of the substrate may be attached to the drum and wound around until the drum(s) may be sufficiently covered with the substrate. The free end of the substrate may then be attached to a drum to keep the substrate from unwinding during rotation of the drum.

### C. Rotational Device

The bioreactor may also include a rotation device to rotate the drum and thereby rotationally expose the biofilm growing on substrate to the growth medium, light, and air. Any suitable rotation device may be employed. The rotational device may comprise a driver, such as a motor or a turbine (in some embodiments driven by hydro or wind forces) and a mechanical attachment, such as a belt, chain, gear assembly, or combination thereof. The rotational device may be mechanically attached the drum, in single or multiple-drum applications. In multiple drum applications, the rotational device may be mechanically linked to multiple drums or multiple rotational devices may be used. The rotational device may further include a controller with optional sensors and/or timers to manually or automatically control the rotational rate of the bioreactor. In some embodiments moisture and/or temperature sensors may be used to ensure that the substrate does not dry out when rotating outside of the medium. The rotational rate may be varied depending on the size of the drum, the distance between drums, or weather conditions.

### D. Harvesting Device

The bioreactor may also include a harvesting device to harvest and collect the biofilm from the substrate.

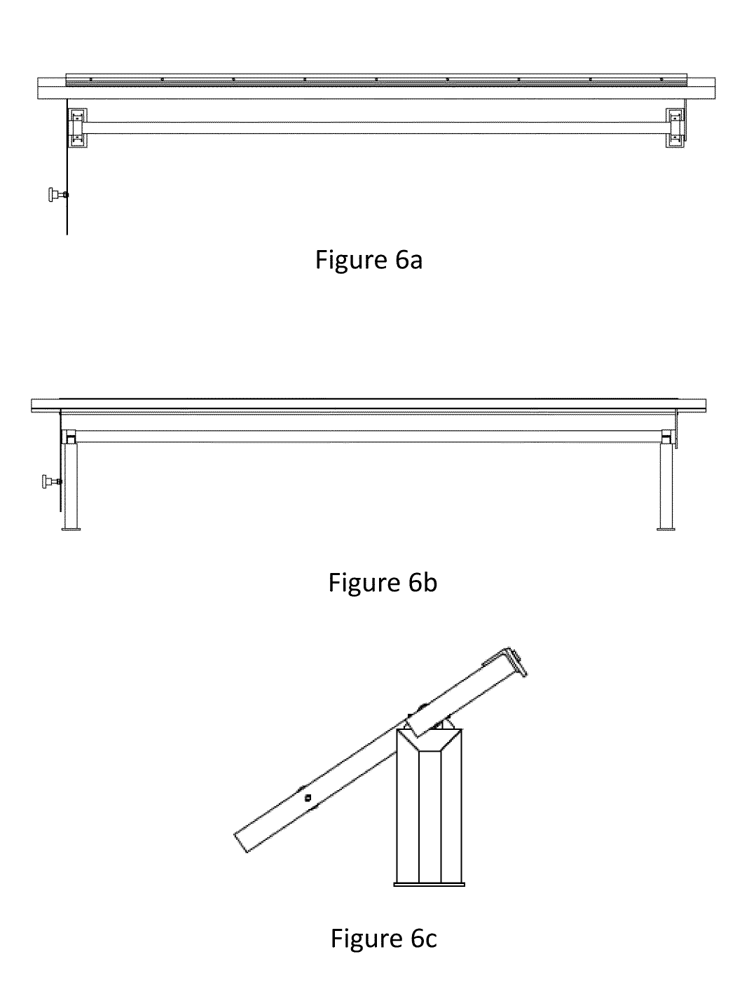

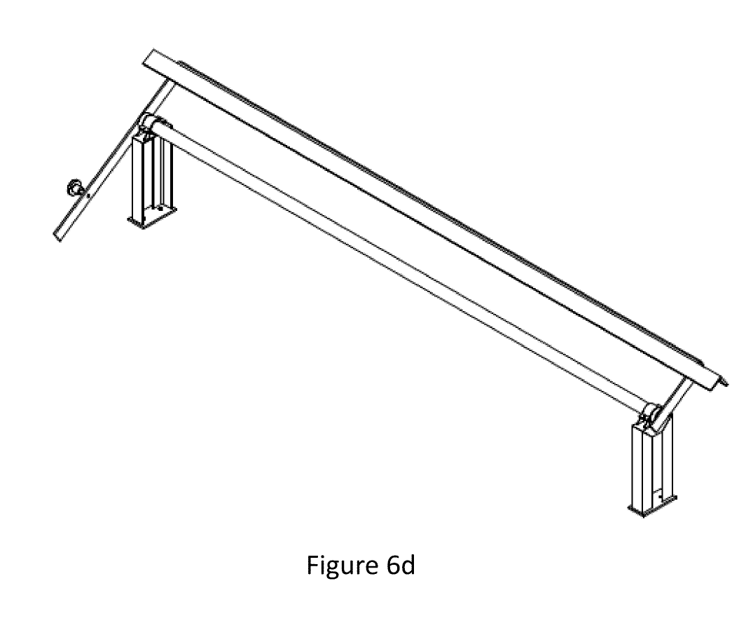

In embodiments employing multiple substrate loops or single coils of substrate, a harvesting device, as illustrated in FIGS. 6a-6d, and a collection device, as illustrated in 7a-7d may be employed. The harvesting device is configured to alternatively engage or disengage contact with the substrate to scrap off at least a portion of the biofilm growing thereon. The harvesting device may have a substantially similar or the same length as the struts on the drum. The harvesting device may be configured to mechanically engage the substrate either by hand or by a driver configured with a controller that is optionally connected to sensors and/or timers. The harvesting device may include a scraper element hingedly connected to mounting base. The harvesting device is configured to be perpendicular to the direction of the substrate.

As with the struts on the drum, the harvesting device may include guide components, as illustrated in FIGS. 4a-4c, so as to guide the harvesting and to engage more area on each of the substrate loops/coils. Thus, in some embodiments, the guide component is the scraper element of the harvesting device. The guide component on the harvesting device may be made of any suitable material, including, for example, metal, alloys, steel, Teflon, Teflon-coated steel, rubber, plastic, composites, or combinations thereof. In embodiments, materials that minimize the wear of the substrate as the guide component scraps off biofilm are preferred.

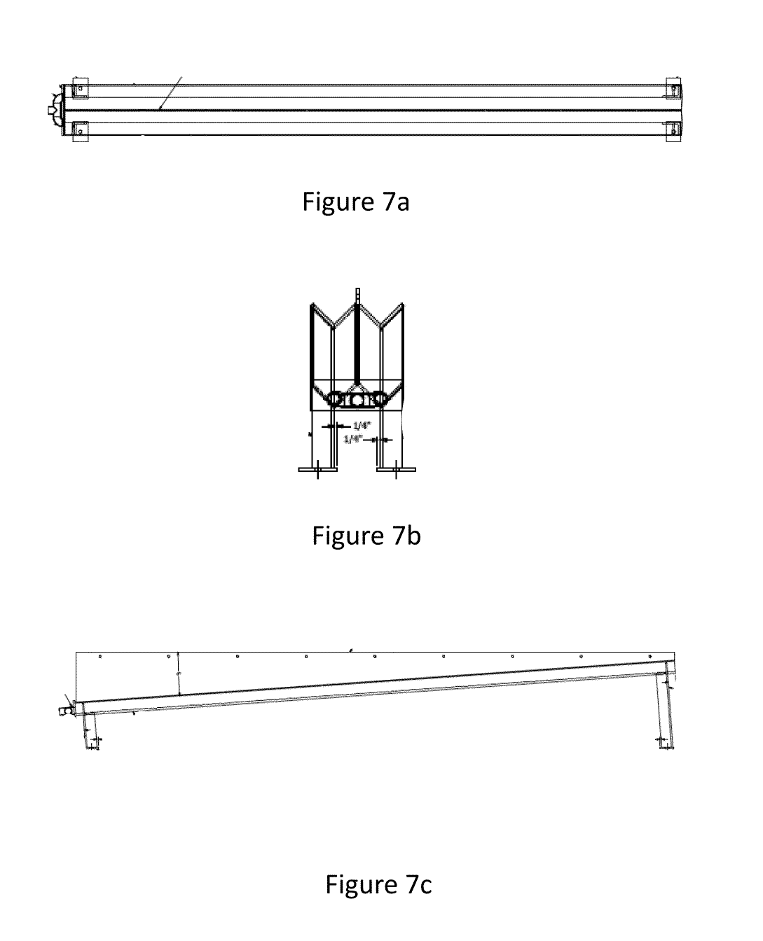

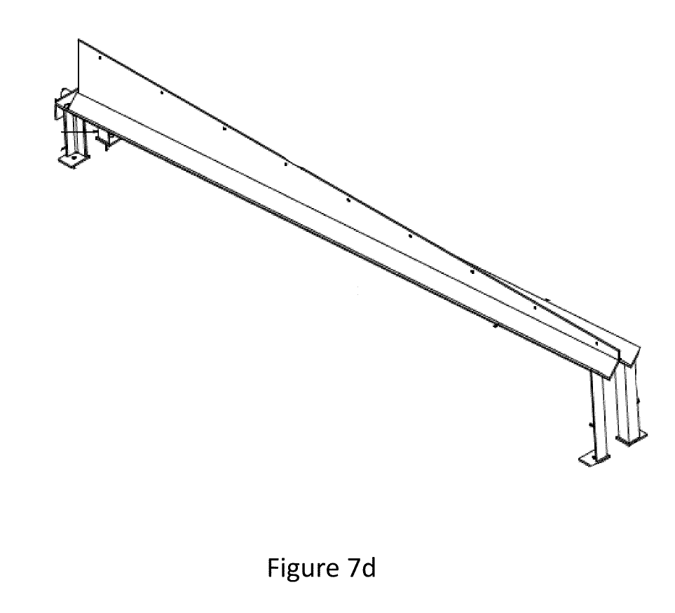

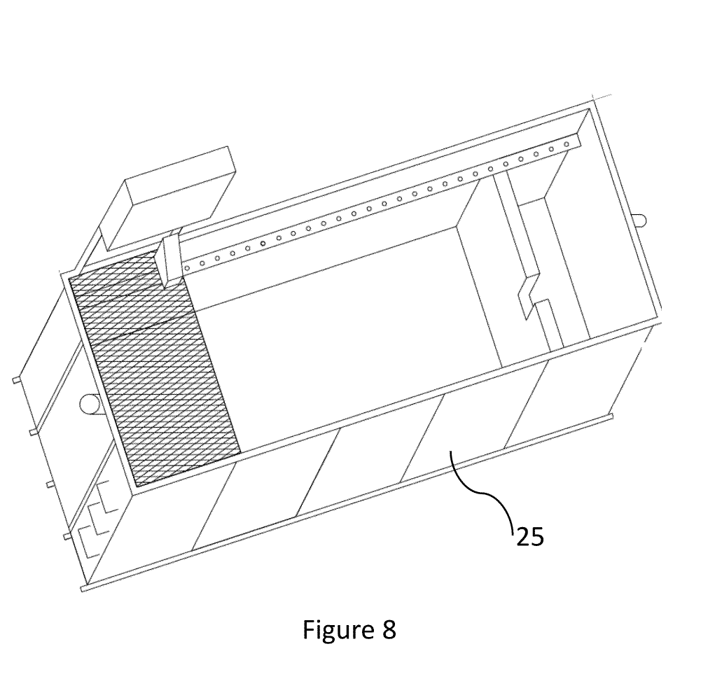

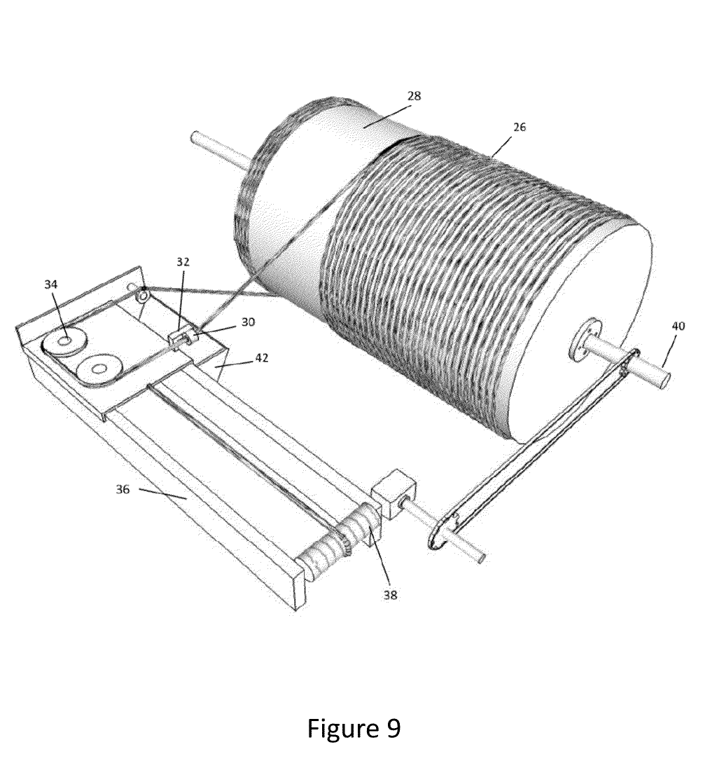

The harvesting device may further comprise a collection device. In embodiments, the collection device is place in a location so that when the scraper element scraps off the biofilm from the substrate, the biofilm falls into a trough, such as the collection device illustrated in FIGS. 7a-7d.

In embodiments having a single coil of substrate, the coil may be threaded through a harvesting device as illustrated in FIG. 9. Referring now to FIG. 9, the biofilm is collected by detaching one end of the substrate 26 from the drum 28 and threading it through a scraper 30. The scraper 30 may be a blade, series of blades, piece of rigid material with a hole in it, or more preferably, a unit configured to have an adjustable diameter and/or constant tension setting like a hose clamp. The scraper 30 may be held in place by attachment to a support 32. A reorientation system 34 is provided to prevent twisting or binding of the substrate 26. The loose end of the substrate 26 is threaded through the scraper 30 and reorientation system 34 until it can be reattached to the drum 28. As the drum 28 continues to rotate, the entire length of the substrate 26 may be pulled through the scraper 30 and pulley system 34 and rewound onto the drum 28. To ensure the substrate 26 may be properly rewound onto the entire length of the drum 28, the scraper 30, support 32, and pulley system 34, are pulled on a support frame 36 along the length of the drum 28 at a rate such that the harvested portion of the substrate 26 may not be layered on top of itself as it is rewound. This may be accomplished with a lateral movement system 38 that may be powered by connection to the drive shaft 40 powering the cylinder 28. Appropriate gear ratios may be chosen to achieve the desired pull rate and spacing of substrate 26. As the biofilm is removed from the substrate 26, it is gathered in a collection bin 42.

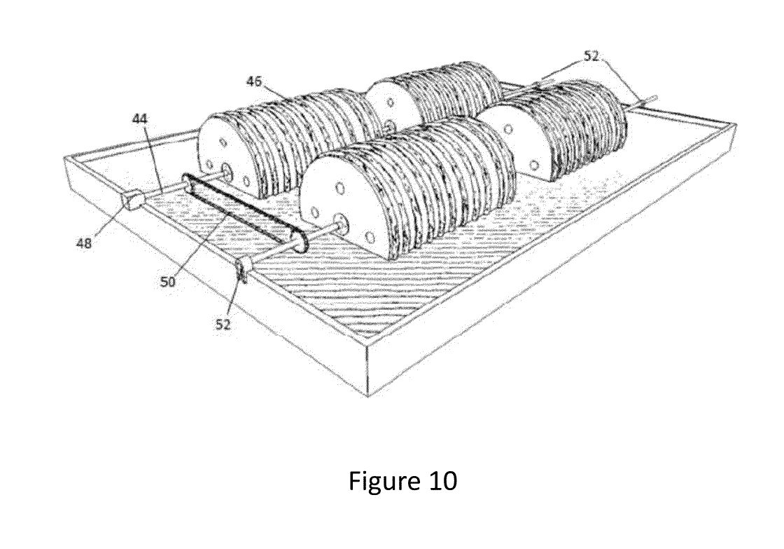

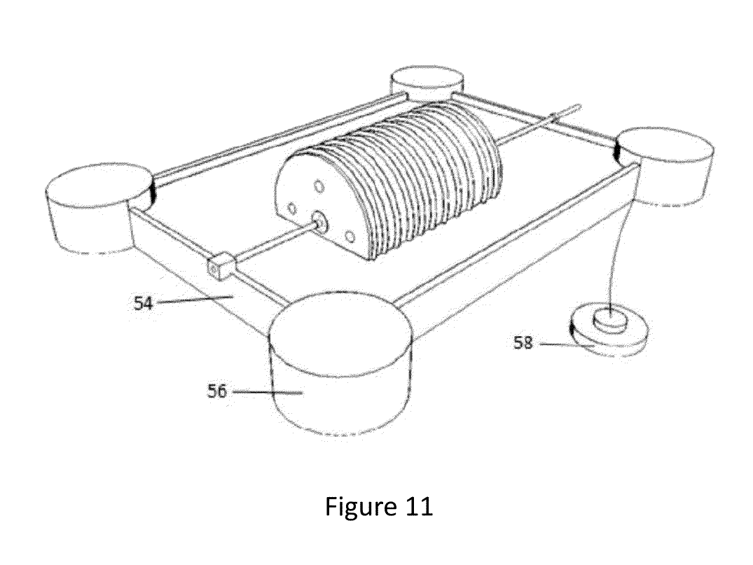

Referring now to another embodiment describing a multiple cylinder setup, shown in FIG. 10, a drive shaft 44 may be made long enough to support two or more cylinders 46 in a train formation. More cylinders 46 may be placed so that rotational power from a motor 48 is transferred to two or more drive shafts 44 through a power transfer mechanism like a roller chain 50. The drive shafts 44 are supported by bearings 52 on each end.

Referring to another embodiment shown in FIG. 11, the entire apparatus may be placed within a support frame 54 with attached floats 56. The apparatus can then be placed at a suitable site and held in place using an anchor 58 or other suitable means of holding it in place. One application of this embodiment of the invention is a retrofitting of oxidation lagoons at a wastewater treatment plant.

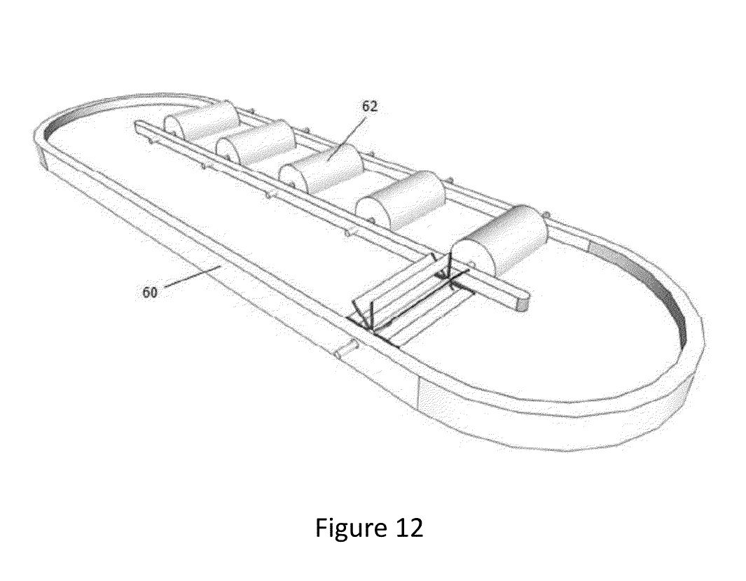

Referring to FIG. 12, another embodiment places the apparatus with a high rate algae pond 60 like a raceway or meandering ditch. The cylinders 62 may be rotated by the force of the passing water or powered by a motor and shaft connected to the cylinder. In a further embodiment, the cylinders 62 may be rotated by an air supply directed at the submerged perimeter of the cylinder in a direction perpendicular to the axis of rotation. In embodiments such as this, the biofilm enhances flocculation of the suspended culture, leading to inexpensive harvesting of all the biomass in the system.

### E. Tank

The bioreactor may be configured in a tank, such as the tank 25 illustrated in FIGS. 1 and 8. In embodiments employing a tank, the tank serves as the base upon which the drum(s), rotational device, and harvesting device are attached. The tank may be configured to include influent and discharge ports so that culture medium may be recharged, drained, or exchanged depending on desired reactor conditions. The tank may also include multiple sections to maximize the growth of biofilm on the substrate, thereby allowing culture medium to enter or exit the tank without striping the biofilm from the submerged substrate. The tank may be made of any suitable material, for example, steel.

In some embodiments, the bioreactor without a tank may be employed. In FIG. 11, a bioreactor is illustrated with a floating partition. Such a design might be used, for example, in a sewage treatment pond. Alternatively, as illustrated in FIG. 12, a raceway design may be employed where at least one bioreactor is placed within a raceway where culture medium is cycled through system.

### EXAMPLES

The following examples are illustrative only and are not intended to limit the disclosure in any way.

### Example 1

A single drum design was designed and tested using three inch diameter PVC pipe that were four feet long. The units sat in a tank system capable of holding 8 liters of media and were submerged 40%. The units were rotated at 4.8 rpm. Eight liters of Logan City Wastewater Effluent was used as media and seed. Eight substrata that demonstrated qualitatively the ability to support algal attachment were tested to demonstrate quantitative data. Nylon, polypropylene, cotton, acrylic, and jute were tested in cord construction, and polyester, high thread cotton, and low thread cotton were in sheet construction. All cord materials except jute were the same diameter.

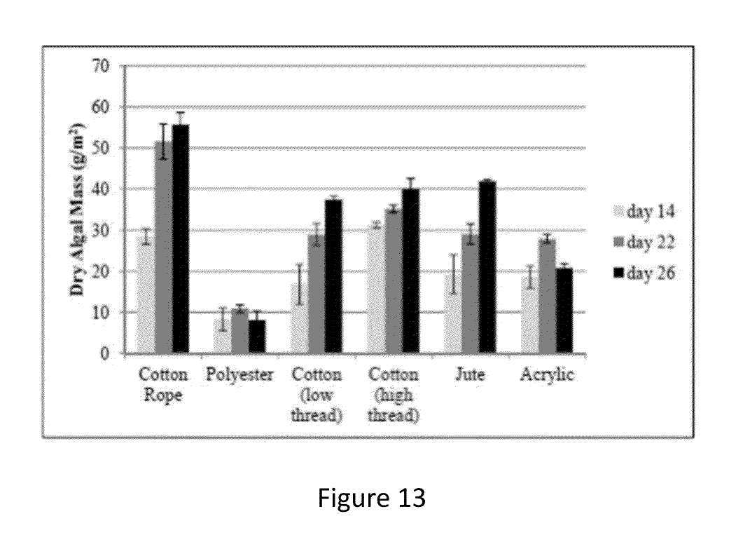

The reactors were operated in fed batch mode with nutrient supplementation every 48 hours to a predetermined nitrogen to phosphorus ratio and concentration. The experiment ran for 26 days after which the solids content on the rope was determined in grams per meter squared. See FIG. 13 Algal biofilms grown on cord substrata were harvested with a scraper mechanism designed to scrape all sides of the cord. The harvesting mechanism used a scraper, pulley system, and the rotational device of the reactors, motors, to operate, as described in FIG. 9.

Subsequent testing of additional substrates were tested with varying levels of thickness, finished surface, surface pliability, hydrophobicity, and durability with the same harvesting mechanism. The testing was conducted similar to the initial testing with the experiment running for 29 days and revalidated the results of the previous testing.

### Example 2

Two tanks were constructed to be 96 inches long, 48 inches wide, and 16 inches deep. One tank was constructed to operate as a raceway pond to support suspended algae growth and the other constructed with a raceway pond with a rotating bioreactor. The rotating bioreactor was constructed using five PVC drums that were 23 inches high and a 16 inch diameter. Cotton cord was used as substrata. The bioreactors were submerged 40% and operated at 5.4 rpm. Each tank was filled with 535 liters of wastewater. Paddlewheels operated at 5.4 rpm within the tanks A specific nitrogen to phosphorus ratio was chosen and maintained. Biomass was measured by solids testing using standard methods. Experiments were conducted for 21 days and demonstrated a dry algal mass of over 350 grams per meter squared for the rotating biofilm reactor versus a biomass concentration of 0.85 grams per liter in the raceway bioreactor with no rotating biofilm reactor.

### Example 3

A pilot scale rotating bioreactor was constructed. The drum and struts were constructed out of aluminum. Two hubs measuring 76 inches in diameter were connected 5 feet apart. Cotton cord was used as the substrata and a tank with a channel measuring 6 feet by 3 feet that contained approximately 8,000 liters of media was used. The media used was effluent from the Logan City, Utah wastewater treatment plant. The reactor operated at 1.2 rpm and had a continuous flow of effluent wastewater supplied at 3 gallons per minute. Biofilms were harvested with a scaper mechanism. Solids content was tested and a biomass production rate of 31 grams per meter squared per day determined.

These tests were repeated in a new tank with dimensions of approximately 8 feet by 8 feet with a volume of 10,000 liters. A variety of different test conditions were implemented over the entire testing period including varying the hydraulic retention time and nutrient supplementation. In all of these the rotating bioreactor design remained the same.

### Example 4

A dual drum design was tested at the pilot scale. The system included two drums, 4 feet in diameter by 7 feet long inside a tank with dimensions of 20 feet by 8 feet by 4 feet. The substratum used was cotton rope with a stainless steel core. The substratum was attached in a coil and wrapped around both drums. The struts on the drums contained grooves to hold the coiled cotton rope in place. See FIG. 4. Two hundred and twelve coils were place on the two drums. The drums were submerged approximately 40% and the rotation of the systems was varied during testing. Biomass was harvested using a scaper system (see FIG. 6) which clamped around each coil and was scaped utilizing the rotational device of the drums, a motor and collected into tank (see FIG. 7). During the experimental operation, an algae biomass yield of 5 to 7 grams per meter squared was observed.

### Example 5

A single drum rotating device in the shape of a star was developed. The hubs measured 74 inches in diameter and were connected by struts measuring 5 feet. The struts were placed into a specific configuration in order for the substrata, cotton rope, to be weaved back and forth with a design that represents a star configuration. The system is situated on top of a tank that is 178 inches by 108 inches by 48 inches. After the initial lag phase, the solids content was shown to vary between 10-30 grams per meter squared sampling three times a week.

The single drum design was tested in wastewater from the Logan City wastewater treatment facility effluent, as well as Bristol media, and produced water. These experiments were conducted with 3 inch diameter PVC, 7 inches long. The system was placed within a tank that was 8 inches by 4.5 inches by 3 inches. The Bristol media testing was compared with wastewater collected from the Logan City wasterwater treatment facility. The system was inoculated with a mixed culture from the Logan City wasterwater treatment facility. Results indicated that in general Bristol media had higher algae biomass yields than the collected wastewater.

Testing of the rotating bioreactor in produced water occurred as described, with several differences. The seed culture utilized was cyanobacteria obtained through previous testing of the rotating bioreactor from the Logan City wastewater treatment facility. Produced water was gathered from a disposal facility in Baggs, Wyo. The experiments were operated in 40 day periods. Quantitative results of the seed culture indicated that it did not grow in solution, only upon an attachment substratum. Experiments demonstrated the ability to grow biomass in produced water as a media source as well as produce biomass that contains valuable by-products. Biomass collected was between 16 to 17 grams per meter squared defined as ash free dry weight.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A bioreactor, comprising:
at least one rotating drum,
a substrate on the drum, configured to grow a biofilm thereon,
a rotational device rotating the drum and substrate, and
a harvesting device.

2. The bioreactor of claim 1, wherein the at least one rotating drum comprises at least two hubs connected by at least two struts.

3. The bioreactor of claim 2, wherein the struts comprise a guide element configured to guide the substrate during rotation.

4. The bioreactor of claim 1, wherein the substrate comprises a coil.

5. The bioreactor of claim 1, wherein the substrate comprises a series of rope loops.

6. The bioreactor of claim 1, wherein the harvesting device comprises a scraper and a collection trough.

7. The bioreactor of claim 6, wherein the scraper comprises a guide element.

8. The bioreactor of claim 1, wherein:
the at least one rotating drum comprises at least two rotating drums,
the substrate comprises a series of loops wrapped around the at least two rotating drums, and
the harvesting device is located between the two rotating drums.

9. The bioreactor of claim 1, further comprising a tank to hold culture medium.

10. A method of cultivating and harvesting algae, the method comprising:
rotating a substrate in and out of a culture medium containing microalgae and nutrients to form an algal biofilm on the substrate,
periodically scraping the substrate to remove at least a portion of the algal biofilm, and collecting the removed algal biofilm.
